(19) United States
(12) Patent Application Publication
(10) Pub. No.: US 2015/0132815 A1
(43) Pub. Date: May 14, 2015

(54) PROCESSES FOR ENHANCING THE PERFORMANCE OF LARGE-SCALE, TANK ANAEROBIC FERMENTORS

(71) Applicant: Robert Hickey, Okemos, MI (US)

(72) Inventor: Robert Hickey, Okemos, MI (US)

(73) Assignee: Coskata, Inc., Warrenville, IL (US)

(21) Appl. No.: 14/599,842

(22) Filed: Jan. 19, 2015

Related U.S. Application Data
(63) Continuation of application No. 13/243,426, filed on Sep. 23, 2011.

(51) Int. Cl. C12P 7/54; C12P 7/06; C12P 7/16; C12P 7/04; C12P 7/52

(52) U.S. Cl. CPC C12P 7/54; C12P 7/04; C12P 7/52; C12P 7/16; C12P 7/065
USPC 435/140; 435/161; 435/157; 435/141; 435/160

(57) ABSTRACT

Processes are disclosed for the low energy, anaerobic bioconversion of hydrogen and carbon monoxide in a gaseous substrate stream to oxygenated organic compounds such as ethanol by contact with microorganisms in a deep, tank fermentation system with high conversion efficiency of both hydrogen and carbon monoxide. Gas feed to the reactor is injected using a motive liquid to form a stable dispersion of microbubbles thereby reducing energy costs, and a portion of the off-gases from the reactor are recycled to (i) achieve a conversion of the total moles of carbon monoxide and hydrogen in the gas substrate to oxygenated organic compound of at least about 80 percent and (ii) attenuate the risk of carbon monoxide inhibition of the microorganism used for the bioconversion.

### CROSS REFERENCE TO RELATED APPLICATION

This application is a continuation of U.S. application Ser. No. 13/243,426 filed Sep. 23, 2011 the disclosure of which are hereby incorporated by reference in its entirety.

### BACKGROUND OF THE INVENTION

1. Field of the Invention

This invention pertains to processes for the low energy, anaerobic bioconversion of hydrogen and carbon monoxide in a gaseous substrate stream to oxygenated organic compounds such as ethanol by contact with microorganisms in a stirred tank fermentation system with high conversion efficiency of both hydrogen and carbon monoxide, and to apparatus for using such processes.

2. Background

Anaerobic fermentations of hydrogen and carbon monoxide involve the contact of the substrate gas in a liquid aqueous menstruum with microorganisms capable of generating oxygenated organic compounds such as ethanol, acetic acid, propanol and n-butanol. The production of these oxygenated organic compounds requires significant amounts of hydrogen and carbon monoxide. For instance, the theoretical equations for the conversion of carbon monoxide and hydrogen to ethanol are:

6CO+3H₂O.C₂H₅OH+4CO₂

6H₂+2CO₂.C₂H₅OH+3H₂O.

As can be seen, the conversion of carbon monoxide results in the generation of carbon dioxide. The conversion of hydrogen involves the consumption of hydrogen and carbon dioxide, and this conversion is sometimes referred to as the H₂/CO₂ conversion. For purposes herein, it is referred to as the hydrogen conversion.

Typically the substrate gas for carbon monoxide and hydrogen conversions is or is derived from a synthesis gas (syngas) from the gasification of carbonaceous materials, reforming of natural gas and/or biogas from anaerobic fermentors or from off streams of various industrial methods. The gas substrate contains carbon monoxide, hydrogen, and carbon dioxide and usually contains other components such as water vapor, nitrogen, methane, ammonia, hydrogen sulfide and the like. (For purposes herein, all gas compositions are reported on a dry basis unless otherwise stated or clear from the context.)

Syngas fermentation processes suffer from the poor solubility of the gas substrate, i.e., carbon dioxide and hydrogen, in the liquid phase of the aqueous menstruum. Munasinghe, et al., in Biomass-derived Syngas Fermentation in Biofuels: Opportunities and Challenges, Biosource Technology, 101 (2010) 5013-5022, summarize volumetric mass transfer coefficients to fermentation media reported in the literature for syngas and carbon monoxide in various reactor configurations and hydrodynamic conditions. A number of conditions can enhance the mass transfer of syngas to the liquid phase. Increasing the interfacial area between the gas feed and the liquid phase can improve mass transfer rates. For stirred tank reactors, they say that increasing the agitation of the impeller improves mass transfer as smaller bubble sizes are obtained. The authors report that in one study, the mass transfer obtained for a bubble column reactor was higher than that for a stirred tank reactor mainly due to the higher interfacial surface area obtained by using a microbubble sparger with the bubble column reactor. They report the findings of another study where it was concluded that the axial mixing of microbubble dispersions in bubble column reactors was considerably less than that of the conventional bubble column reactors. Munasignhe, et al., in a later published paper, Syngas Fermenation to Biofuel: Evaluation of Carbon Monoxide Mass Transfer Coefficent (k_{L}a) in Different Reactor Configurations, Biotechol. Prog., 2010, Vol. 26, No. 6, pp 1616-1621, combine a sparger (0.5 millimeter diameter pores) with mechanical mixing at various rotational rates to provide enhanced mass transfer. They also report on prior work by others who used a stirred tank and microbubble sparger to obtain high volumetric mass transfer coefficients.

Bredwell, et al., in Reactor Design Issues for Synthesis-Gas Fermentations, Biotechnol. Prog., 15 (1999) 834-844, assessed various types of reactors including bubble columns and stirred reactors. The authors disclose using microbubble sparging with mechanical agitation. At page 839 they state:

“When microbubble sparging is used, only enough power must be applied to the reactor to provide adequate liquid mixing. Thus axial flow impellers designed to have low shear and a high pumping capacity would be suitable when microbubbles are used in stirred tanks.”
They conclude by stating:
“An improved ability to predict and control coalescence rates is needed to rationally design commercial-scale bioreactors that employ microbubble sparging.”

For a syngas to oxygenated organic compound fermentation process to be commercially viable, capital and operating costs must be sufficiently low that it is at least competitive with alternative biomass to oxygenated organic compound processes. For instance, ethanol is commercially produced from corn in facilities having name plate capacities of over 100 million gallons per year. Accordingly, the syngas to oxygenated organic compound fermentation process must be able to take advantage of similar economies of scale. Thus, a commercial scale facility may require at least 20 million liters of fermentation reactor capacity. As problems with stirred tank reactors are capital costs, the significant amount of energy needed for gas transfer and mixing, and the need for plural stages to achieve high conversion of gaseous substrates, stirred tank reactors face considerable difficulties in being justified for these commercial-scale facilities. Reported by Munasignhe, et al., other syngas fermentation reactor types such a bubble column reactors and air lift (jet loop) reactors are less costly to manufacture and operate yet can provide good mass transfer rates of syngas to the liquid phase. However, microbubble spargers, especially for very small microbubbles, use significant amounts of energy and are prone to fouling. Accordingly, other means for generating microbubbles such as injectors using a motive fluid that are not prone to fouling, are preferred. Co-pending U.S. patent application Ser. No. 12/826,991, filed on Jun. 30, 2010, herein incorporated by reference in its entirety, discloses the use of injectors to supply gas feed to an anaerobic fermentation in a deep reactor to make a liquid product such as ethanol wherein the presence of the liquid product enables the injector to produce a dispersion of microbubbles.

In addition to economies of scale, the processes need to obtain high conversion efficiencies of the syngas to oxygenated organic compounds. Syngas and other carbon monoxide and hydrogen-containing gas feeds are typically more expensive than equivalent heat content amounts of fossil fuels. Hence, a desire exists to use these gases effectively to make higher value products. The financial viability of any conversion process, especially to commodity chemicals such as ethanol and acetic acid, will be dependent upon the efficiency of conversion of the carbon monoxide and hydrogen, the selectivity of conversion to the sought products and the energy costs to effect the conversion.

Accordingly, a multitude of challenges are faced when seeking to take advantage of the benefits of stirred tank reactors for the conversion of syngas to oxygenated organic compound at the large scale required for commercial viability. In their review article, Munasignhe, et al., report that the mass transfer coefficient for slightly soluble gaseous substrates is dependent upon the difference in partial pressures in the gas and in the liquid phases. The authors state at page 5017:

“High pressure operation improves the solubility of the gas in the aqueous phase. However, at higher concentrations of gaseous substrates, especially CO, anaerobic microorganisms are inhibited.”

Other workers have understood that the presence of excess carbon monoxide can adversely affect the microorganisms and their performance. See paragraphs 0075 through 0077 and 0085 though 0086 of United States published patent application No. 20030211585 (Gaddy, et al.) disclosing a continuously stirred tank bioreactor for the production of ethanol from microbial fermentation. At paragraph 0077, Gaddy, et al., state:

“The presence of excess CO unfortunately also results in poor H₂ conversion, which may not be economically favorable. The consequence of extended operation under substrate inhibition is poor H₂ uptake. This eventually causes cell lysis and necessary restarting of the reactor. Where this method has an unintended result of CO substrate inhibition (the presence of too much CO for the available cells) during the initial growth of the culture or thereafter, the gas feed rate and/or agitation rate is reduced until the substrate inhibition is relieved.”

At paragraph 0085, Gaddy, et al., discuss supplying excess carbon monoxide and hydrogen. They state:

“A slight excess of CO and H₂ is achieved by attaining steady operation and then gradually increasing the gas feed rate and/or agitation rate (10% or less increments) until the CO and H₂ conversions just start to decline.”

Thus a commercial-scale process using a stirred tank reactor must be able to balance obtaining desirable rates of diffusion of carbon monoxide into the aqueous menstruum with avoiding carbon monoxide inhibition.

Accordingly, commercial-scale processes are sought to take advantage of the mixing provided by stirred tank reactors without undue capital and operating costs while achieving high conversion of gas substrate, selectivity to oxygenated organic compound and avoiding carbon monoxide inhibition. Moreover, processes are sought that enable effective process control.

### SUMMARY OF THE INVENTION

By this invention, a single, commercial-scale, continuous stirred tank reactor is able to achieve high bioconversion of gas substrate comprising carbon monoxide and hydrogen to oxygenated organic compound by anaerobic fermentation in an aqueous menstruum without undue energy costs. Commercial viability is further enhanced as preferred processes of this invention can provide high conversions to oxygenated organic compound employing vessels rated for use essentially at atmospheric pressure. The processes of this invention use a deep, stirred tank reactor having a height of at least about 10, often between about 10 or 15 and 30, meters and an aspect ratio of height to diameter of at least about 0.5:1, say, 0.5:1 to 5:1, preferably between about 0.75:1 to 3:1, and a width of at least about 5, preferably at least about 7, and often between about 7 and 30, meters. The processes of this invention use a relatively stable gas-in-water dispersion in the aqueous menstruum which dispersion is generated by injection of the gas feed with a motive liquid. The processes of this invention further comprise recycling a portion of the off-gas from the aqueous menstruum back to the aqueous menstruum in admixture with fresh gas feed in an amount sufficient to (i) achieve a molar conversion of the total of carbon monoxide and hydrogen in the gas feed to oxygenated organic compound of at least about 80, preferably at least about 85, often between about 85 and 95, percent and (ii) attenuate the risk of carbon monoxide inhibition.

Importantly, by recycling of a portion of the off-gas for admixture with fresh gas feed being passed to the reactor, the composition of the gas bubbles can be adjusted such that the rate of mass transfer to the aqueous menstruum does not unduly exceed the rate of bioconversion of carbon monoxide and thereby avoid carbon monoxide inhibition. Since the processes of this invention achieve high conversion efficiencies of carbon monoxide and hydrogen, the off-gases at steady state operating conditions will have a low mole fraction of carbon monoxide and hydrogen and thus be effective for controlling the composition of the gas bubbles being passed to the reactor. Moreover, the recycling of a portion of the off-gas enables microbubbles to be generated that result in a stable gas-in-liquid dispersion. By providing a stable gas-in-liquid dispersion, predictive control of the transfer of carbon monoxide and hydrogen to the aqueous menstruum is facilitated thereby enabling high conversions of carbon monoxide and hydrogen to oxygenated product to be achieved without incurring undue risk of carbon monoxide inhibition.

The stirred tank reactor uses one or more mechanical stirrers and provides a beneficial ratio of energy for mechanical stirring to volume. Preferably the mechanical stirring is at a rate insufficient to cause undue agglomeration of gas phase microbubbles. The mechanical stirring should be sufficient to promote the uniformity of liquid composition through the reactor and need not, and preferably is not, used as a generator of a significant fraction of the microbubbles. The mechanical stirring can be performed by any suitable means. Due, however, to the large volume of the tank reactor, the mechanical stirring is preferably conducted using side paddles or blades or side-mounted impellers. Paddles are a preferred mechanical stirrer due to the liquid circulation rate that can be achieved with low energy consumption and low speeds that minimize coalescence of the microbubbles. For purposes herein the type of stirred tank reactor used in the processes of this invention is called a mechanically-assisted liquid distribution tank reactor, or MLD tank reactor. With the relatively uniform composition throughout the MLD tank reactor provided by the mechanical stirring, regardless of where the gas feed is introduced, microbubbles will be moved through out the volume of the aqueous menstruum.

By using a motive fluid for instance in a venturi or jet injector, to generate the microbubbles for the dispersion, rather than the mechanical stirring, energy savings are realized. Moreover, the injectors can provide better control over the size of the gas bubbles being introduced into the aqueous menstruum and thus the interfacial area between the gas and liquid phases. Changing bubble size thus modulates the mass transfer of carbon monoxide and hydrogen to the aqueous menstruum. Additionally, the modulation enables a microbubble size to be generated that results in a preferred, stable gas-in-water dispersion. Since the mechanical stifling does not adversely affect the gas bubbles, the modulation achieved by adjusting bubble size provides a viable control of the process.

In its broad aspect, the processes of this invention comprise the anaerobic bioconversion of a gas substrate comprising carbon monoxide and hydrogen in an aqueous menstruum containing microorganisms suitable for bioconverting said substrate to oxygenated organic compound in a deep, continuously-stirred tank reactor comprising:

a. maintaining under continuous mechanical stirring in a reactor an aqueous menstruum containing said microorganisms, said aqueous menstruum being under anaerobic fermentation conditions, and said aqueous menstruum having an upper portion with a head space above the upper portion and a lower portion and having a depth of at least 10 meters in said reactor; and
b. continuously supplying gas feed comprising said gas substrate to said aqueous menstruum by injection using in a motive liquid to form a stable gas-in-liquid dispersion in the aqueous menstruum, bioconverting carbon monoxide and hydrogen and carbon dioxide to oxygenated organic compound and providing off-gas from the aqueous menstruum in the head space,
c. withdrawing from the head space of said reactor at least a portion of the off-gas; and
d. admixing at least a portion of the withdrawn off-gas with the gas substrate in an amount sufficient to (i) achieve a bioconversion efficiency of the total moles of carbon monoxide and hydrogen in the gas substrate to oxygenated organic compound of at least about 80 percent and (ii) attenuate the risk of carbon monoxide inhibition of the microorganism used for the bioconversion
wherein the mechanical stifling is sufficient to provide relatively uniform liquid phase composition within the aqueous menstruum without unduly adversely affecting the gas-in-liquid dispersion. Often, the energy required for the mechanical stirring is less than 0.02 watt per liter of aqueous menstruum.

The stable gas-in-water dispersion is provided using microbubbles of gas, preferably less than 500, more preferably less than 300, say about 10 or 20 to 300, microns in diameter. Suitable devices for generating and introducing the gas-in-liquid dispersions include venturi injectors, jet injectors and, preferably, slot injectors, where the motive liquid contains oxygenated organic compound or other surface active agent. As the size of the microbubbles can be varied by changing the rate of flow of the motive liquid, an additional means for control of the mass transfer of gas substrate to the liquid phase can be achieved. Jet injectors, and especially slot injectors, can provide a suitable sized bubble to enable the stable dispersion to be formed while maintaining a surface area to volume ratio to provide a rate of transfer high enough to obtain desired efficiencies of conversion but low enough to avoid carbon monoxide inhibition.

In a preferred aspect of the invention, the rate of supply of fresh gas feed for admixing with recycled off-gas is controlled in response to the conversion efficiency. In this aspect, the rate that carbon monoxide and hydrogen transfer to the liquid phase can readily be adjusted to reflect the conditions of the aqueous menstruum, thereby optimizing conversion of gas substrate while avoiding the risk of carbon monoxide inhibition. The rate of transfer of carbon monoxide to the liquid phase would therefore be in concert with the rate that the culture of microorganisms can bioconvert the carbon monoxide, i.e., no build-up of carbon monoxide concentration would occur in the aqueous menstruum.

The motive liquid may be any suitable aqueous liquid for introduction into the aqueous menstruum including make-up water, aqueous streams from product recovery, aqueous streams recovered from the purge of solids and recycled aqueous menstruum. In a preferred aspect of the invention, at least a portion of the motive, aqueous liquid is derived from aqueous menstruum withdrawn from the reactor. In one embodiment, the introduction of gas feed is accomplished at a lower portion of the reactor and aqueous menstruum for recycle is withdrawn from an upper portion of the reactor. Although the composition of the aqueous menstruum is relatively uniform throughout the reactor, this embodiment takes advantage of relatively small compositional differences. In other embodiments, the gas feed is supplied at two or more heights in the reactor.

The invention also pertains to apparatus for anaerobic bioconversion of a gas substrate comprising carbon monoxide and hydrogen in an aqueous menstruum containing microorganisms suitable for converting said substrate to oxygenated organic compound comprising:

a. a deep, continuously-stirred tank reactor having a height of at least 10 meters and at least one mechanical stirrer, said tank reactor being adapted to contain under anaerobic fermentation conditions an aqueous menstruum and defining a head space adapted to receive off-gas from the aqueous menstruum;
b. at least one injector in the tank reactor adapted to provide a gas-in-liquid dispersion;
c. a gas feed supply line in fluid communication with said injector adapted to provide fresh gas feed containing said substrate;
d. a motive liquid supply line adapted to provide liquid to said injector, said motive liquid supply line being in fluid communication with the stirred tank reactor to obtain at least a portion of the motive liquid to said injector;
e. an off-gas exhaust line from the head space of the tank reactor; and
f. a recycle off-gas line in fluid communication between the off-gas exhaust line and the gas feed supply line adapted to provide recycle off-gas for admixture with gas substrate.

Preferably the apparatus further comprises:

g. a control processor in communication with a gas analyzer in fluid communication with the head space and adapted to determine the concentration of carbon monoxide and hydrogen in the off-gas from the aqueous menstruum and with a flow meter adapted to determine the flow rate of off-gas being produced, said control processor adapted to determine the conversion efficiency of carbon monoxide and hydrogen in the tank reactor; and
h. a valve in the gas feed supply line adapted to control the rate of flow of fresh gas feed in response to the determination by the control processor of conversion efficiency of carbon monoxide and hydrogen.
Preferably the apparatus use at least two mechanical stirrers at different heights in the tank reactor.

As used herein, the article “a” is not intended to restrict the apparatus to containing only one of the designated element, and is to be interpreted as meaning that the apparatus contains at least one of the designated element.

### BRIEF DESCRIPTION OF THE DRAWINGS

FIG. 1 is a schematic flow diagram of a deep, MLD tank reactor adapted to use the process of this invention.

### DETAILED DESCRIPTION OF THE INVENTION

### Definitions

Oxygenated organic compound means one or more organic compounds containing two to six carbon atoms selected from the group of aliphatic carboxylic acids and salts, alkanols and alkoxide salts, and aldehydes. Often oxygenated organic compound is a mixture of organic compounds produced by the microorganisms contained in the aqueous menstruum.

Uniformity in the liquid phase means that the composition of the aqueous menstruum is relatively uniform throughout the deep, MLD reactor. Uniformity can be determined by measuring the concentration of oxygenated organic compound in samples taken at a lower portion and at an upper portion of the aqueous menstruum, and uniformity exists if the concentration of the oxygenated organic compound in the samples does not vary by more than 20 mole percent.

Aqueous menstruum means a liquid water phase which may contain dissolved compounds including, but not limited to hydrogen, carbon monoxide, and carbon dioxide.

The motive liquid may be any suitable liquid for introduction into the reactor. The motive liquid comprises sufficient amount of one or more of oxygenated organic compound and other surface active agent to enhance the formation of microbubbles.

Microbubbles are bubbles having a diameter of 500 microns or less.

The pressure at the point of injection into the aqueous menstruum is the sum of the absolute pressure at the point calculated as if the liquid head above such point were water. The partial pressure of a gas feed component is determined as the product of the mole fraction of a component in a gas mixture times the total pressure. The partial pressure of a component in the gas being fed to a reaction reactor is calculated as the mole fraction of that component times the pressure in the reaction reactor at the point of entry.

Stable gas-in-liquid dispersion means a mixture of gas bubbles in liquid where (i) the bubbles predominantly flow in the same direction as the liquid, and (ii) the dispersion is sufficiently stable that it exists throughout the aqueous menstruum, i.e., insufficient coalescing of bubbles occurs to destroy the dispersion.

Carbon monoxide inhibition means that microorganisms are adversely affected by a high concentration of dissolved carbon monoxide in the aqueous menstruum resulting in a significantly reduced, e.g., reduced by at least 15 percent, conversion of carbon monoxide or hydrogen per gram of active cells per liter, all other conditions remaining the same. The inhibitory effect may occur in a localized region in the aqueous menstruum; however, the occurrence of a carbon monoxide inhibition is typically observed by assessing the specific activity rate, i.e., the mass bioconsumed per mass of active microorganism per unit time, which under steady-state conditions can be approximated by the overall conversion for the volume of aqueous menstruum in the reactor. The concentration of carbon monoxide dissolved in the aqueous menstruum that results in carbon monoxide inhibition varies depending upon the strain of microorganism and the fermentation conditions.

### Overview:

The processes of this invention pertain to operating deep, stirred tank fermentation reactors, particularly deep, MLD tank reactors, for anaerobic conversion of gas substrate containing carbon monoxide, hydrogen and carbon dioxide to produce oxygenated organic compound such as ethanol, acetic acid, propanol, propionic acid, butanol and butyric acid.

### Substrate and Feed Gas:

Anaerobic fermentation to produce oxygenated organic compound uses a substrate comprising carbon monoxide, carbon dioxide and hydrogen, the later being for the hydrogen conversion pathway. The gas feed will typically contain nitrogen and methane in addition to carbon monoxide and hydrogen. Syngas is one source of a gas substrate. Syngas can be made from many carbonaceous feedstocks. These include sources of hydrocarbons such as natural gas, biogas, biomass, especially woody biomass, gas generated by reforming hydrocarbon-containing materials, peat, petroleum coke, coal, waste material such as debris from construction and demolition, municipal solid waste, and landfill gas. Syngas is typically produced by a gasifier. Any of the aforementioned biomass sources are suitable for producing syngas. The syngas produced thereby will typically contain from 10 to 60 mole % CO, from 10 to 25 mole % CO₂ and from 10 to 60 mole % H₂. The syngas may also contain N₂ and CH₄ as well as trace components such as H₂S and COS, NH₃ and HCN. Other sources of the gas substrate include gases generated during petroleum and petrochemical processing. These gases may have substantially different compositions than typical syngas, and may be essentially pure hydrogen or essentially pure carbon monoxide. The gas substrate may be obtained directly from gasification or from petroleum and petrochemical processing or may be obtained by blending two or more streams. Also, the gas substrate may be treated to remove or alter the composition including, but not limited to, removing components by chemical or physical sorption, membrane separation, and selective reaction. Components may be added to the gas substrate such as nitrogen or adjuvant gases such as ammonia and hydrogen sulfide.

For the sake of ease of reading, the term syngas will be used herein and will be intended to include these other gas substrates.

### Oxygenated Compounds and Microorganisms:

The oxygenated organic compounds produced in the processes of this invention will depend upon the microorganism used for the fermentation and the conditions of the fermentation. Bioconversions of CO and H₂/CO₂ to acetic acid, n-butanol, butyric acid, ethanol and other products are well known. For example, in a recent book concise description of biochemical pathways and energetics of such bioconversions have been summarized by Das, A. and L. G. Ljungdahl, Electron Transport System in Acetogens and by Drake, H. L. and K. Kusel, Diverse Physiologic Potential of Acetogens, appearing respectively as Chapters 14 and 13 of Biochemistry and Physiology of Anaerobic Bacteria, L. G. Ljungdahl eds., Springer (2003). Any suitable microorganisms that have the ability to convert the syngas components: CO, H₂, CO₂ individually or in combination with each other or with other components that are typically present in syngas may be utilized. Suitable microorganisms and/or growth conditions may include those disclosed in U.S. patent application Ser. No. 11/441,392, filed May 25, 2006, entitled “Indirect Or Direct Fermentation of Biomass to Fuel Alcohol,” which discloses a biologically pure culture of the microorganism Clostridium carboxidivorans having all of the identifying characteristics of ATCC no. BAA-624; U.S. Pat. No. 7,704,723 entitled “Isolation and Characterization of Novel Clostridial Species,” which discloses a biologically pure culture of the microorganism Clostridium ragsdalei having all of the identifying characteristics of ATCC No. BAA-622; both of which are incorporated herein by reference in their entirety. Clostridium carboxidivorans may be used, for example, to ferment syngas to ethanol and/or n-butanol. Clostridium ragsdalei may be used, for example, to ferment syngas to ethanol.

Suitable microorganisms and growth conditions include the anaerobic bacteria Butyribacterium methylotrophicum, having the identifying characteristics of ATCC 33266 which can be adapted to CO and used and this will enable the production of n-butanol as well as butyric acid as taught in the references: “Evidence for Production of n-Butanol from Carbon Monoxide by Butyribacterium methylotrophicum,” Journal of Fermentation and Bioengineering, vol. 72, 1991, p. 58-60; “Production of butanol and ethanol from synthesis gas via fermentation,” FUEL, vol. 70, May 1991, p. 615-619. Other suitable microorganisms include: Clostridium Ljungdahlii, with strains having the identifying characteristics of ATCC 49587 (U.S. Pat. No. 5,173,429) and ATCC 55988 and 55989 (U.S. Pat. No. 6,136,577) that will enable the production of ethanol as well as acetic acid; Clostridium autoethanogemum sp. nov., an anaerobic bacterium that produces ethanol from carbon monoxide. Jamal Abrini, Henry Naveau, Edomond-Jacques Nyns, Arch Microbiol., 1994, 345-351; Archives of Microbiology 1994, 161: 345-351; and Clostridium Coskatii having the identifying characteristics of ATCC No. PTA-10522 filed as U.S. Ser. No. 12/272,320 on Mar. 19, 2010. All of these references are incorporated herein in their entirety.

### Aqueous Menstruum and Fermentation Conditions:

The aqueous menstruum will comprise an aqueous suspension of microorganisms and various media supplements. Suitable microorganisms generally live and grow under anaerobic conditions, meaning that dissolved oxygen is essentially absent from the fermentation liquid. The various adjuvants to the aqueous menstruum may comprise buffering agents, trace metals, vitamins, salts etc. Adjustments in the menstruum may induce different conditions at different times such as growth and non-growth conditions which will affect the productivity of the microorganisms. Previously referenced U.S. Pat. No. 7,704,723 discloses the conditions and contents of suitable aqueous menstruum for bioconversion CO and H₂/CO₂ using anaerobic microorganisms.

The top of the deep, MLD tank reactor may be under pressure, at atmospheric pressure, or below ambient pressure. Preferably the fermentation is conducted at substantially atmospheric pressure, for instance with a pressure at the top of less than about 30 kPa gauge, to reduce capital cost of the reactor. The menstruum is maintained under anaerobic fermentation conditions including a suitable temperature, say, between 25 C and 60 C, frequently in the range of about 30 C to 40 C. The conditions of fermentation, including the density of microorganisms, aqueous menstruum composition, and aqueous menstruum depth, are preferably sufficient to achieve the sought conversion of hydrogen and carbon monoxide.

The average residence time of the gas in the fermentation zone will depend upon the depth of the aqueous menstruum and the size of the microbubbles and the internal fluid flows in the vessel cause by the mechanical stirring. While baffles or other flow-directing devices can be used, they are not essential to this invention. In general, the average residence time is between about 50 and 1000, say 100 and 300, seconds.

Any suitable procedure may be used to start-up a deep, MLD tank reactor. Typically, the reactor is filled with a gas not containing reactive oxygen. Although a wide variety of gases for blanketing can be used, such as gases containing carbon dioxide, nitrogen or lower alkane, e.g., alkane of 1 to 3 carbon atoms such as methane and natural gas, cost and availability considerations play a role in the selection of the blanketing gas as well as its acceptability to the anaerobic fermentation process and subsequent unit operations. The reactor is partially charged with aqueous menstruum containing microorganisms and gas feed is provided to grow the culture of microorganisms and additional aqueous menstruum is provided until the aqueous menstruum has obtained the desired height in the reactor and the density of microorganisms has reached its desired level. Start-up procedures for deep tank reactors are disclosed in U.S. Pat. No. 8,936,927 and herein incorporated by reference in its entirety.

Deep, MLD Tank Reactors and their Operation

The deep, MLD tank reactor is of a sufficient volume that the fermentation process is commercially viable. Preferably the reactors are designed to contain at least 1 million, and more preferable at least about 5, say about 5 to 25 million, liters of aqueous menstruum. The reactors are characterized as having a height of at least about 10, often between about 10 or 15 and 30, meters and an aspect ratio of height to diameter of at least about 0.5:1, say, 0.5:1 to 5:1, preferably between about 0.75:1 to 3:1. The commercial-scale reactors are also characterized by a width of at least about 5, preferably at least about 7, and often between about 7 and 30, meters. While the reactors are typically circular in cross-section, other cross-sectional configurations can be used provided that uniformity in the liquid phase is obtained. The height of the aqueous menstruum will establish a hydrostatic pressure gradient along the axis of the reactor.

The deep, MLD tank reactors use one or more mechanical stirrers. The mechanical stirring should be sufficient to promote the uniformity of liquid composition through the reactor and need not, and preferably is not, used as a generator of a significant fraction of the microbubbles. Usually two or more mechanical stirrers are used at different heights with higher aspect ratio reactors. The design of mechanical stirrers for stirred tank reactors and their positioning within the reactors for very large diameter tanks are well within the skill of a stirred tank reactor designer. Side paddles or side mounted mixers with impellers are frequently used. Preferably the design of the mechanical stirrers and the positioning within the reactor take into consideration energy costs in generating the liquid flow to obtain uniformity of the aqueous menstruum in the reactor. The deep, MLD tank reactor may contain baffles or other static flow directing devices.

The depth of the aqueous menstruum in the deep, MLD tank reactor will occupy either the full height or nearly the full height of the reactor. The height of the aqueous menstruum will establish a hydrostatic pressure gradient along the reactor. The dispersion of gas and liquid in the dispersion stream must overcome this hydrostatic pressure at the point where it enters the reactor. Thus if the gas feed enters at a point of 10 meters below the liquid surface the static pressure head inside the vessel would equal approximately 100 kPa gauge and for a liquid height of 15 meters the static pressure head would equal approximately 150 kPa gauge.

### Gas Feed Supply and Injection:

The rate of supply of the gas feed under steady state conditions to each of the primary and sequential reactors is such that the rate of transfer of carbon monoxide and hydrogen to the liquid phase matches the rate that carbon monoxide and hydrogen are bioconverted. Hence, the dissolved concentration of carbon monoxide and hydrogen in the aqueous phase remains constant, i.e., does not build-up. The rate at which carbon monoxide and hydrogen can be consumed will be affected by the nature of the microorganism, the concentration of the microorganism in the aqueous menstruum and the fermentation conditions. As the rate of transfer of carbon monoxide and hydrogen to the aqueous menstruum is a parameter for operation, conditions affecting the rate of transfer such as interfacial surface area between the gas and liquid phases and driving forces are important. For instance, at a given flow rate of gas feed having a given composition to a reactor, the rate of transfer of carbon monoxide and hydrogen can vary widely depending upon the size of the microbubbles and upon the pressure. As discussed below, the processes of this invention supply gas feed by injection using a motive fluid. Variations in the motive liquid flow rate can be used to modulate the microbubble size and thus modulate the rate of transfer of carbon monoxide and hydrogen to the liquid phase. Moreover, the modulation provides microbubbles that provide a stable gas-in-liquid dispersion.

The processes of this invention use at least one injector using a motive fluid for supplying gas feed to the aqueous menstruum. Preferably the reactor contains 2 or more injectors, and commercial scale reactors will often contain at least 2, often 4 to 8 or 10, laterals of injectors with as many as 100 or more injectors. The number of injectors used is typically selected based upon the ability to be able to transfer adequate amounts of gas substrate under steady-state operating conditions and to enhance cross-sectional uniformity of the gas phase in the reactor.

The injectors may be jet mixers/aerators or slot injectors. Slot injectors are preferred, one form of which is disclosed in U.S. Pat. No. 4,162,970. These injectors operate using a motive liquid. The injectors, especially slot injectors, are capable of operating over a wide range of liquid and gas flow rates and thus are capable of significant turn down in gas transfer capability. The injectors are characterized as having nozzles of at least about 1, often about 1.5 to 5, say, 2 to 4, centimeters as the cross-sectional dimension in the case of jet injectors or as the smaller cross-sectional dimension in the case of slot injectors. The large cross-sectional dimension of the injectors provides several benefits in addition to being able to produce microbubbles. First, they are not prone to fouling including where aqueous menstruum is used as the motive liquid as would be a sparger designed to produce microbubbles. Second, where the aqueous menstruum is used as the motive fluid, high momentum impact of the microorganisms with solid surfaces is minimized thereby minimizing the risk of damage to the microorganisms. Third, the energy required to provide microbubbles of a given size is often less than that required to form microbubbles of that size using a microbubble sparger. Fourth, a high turn down ratio can be achieved. And fifth, the microbubble size can be easily varied over a wide range.

The bubble size generated by the injectors will be influenced by, among other factors, the rate of liquid flow through the injector and the ratio of gas phase to liquid phase passing through the injector as well as characteristics of the aqueous menstruum itself including, but not limited to its static liquid depth. Consequently, an injector can be operated to provide a selected bubble size which enhances the ability to use the injector in a modulation mode, i.e., provide the adjustment in the rate of transfer of carbon monoxide to the liquid phase based upon the size of the culture and its ability of the culture to bioconvert the carbon monoxide. The modulation can be obtained by changing one or more of (i) the gas to liquid flow ratio to the injector thus changing the volume of gas feed and (ii) changing the rate of motive liquid and thus the bubble size which affects the rate of transfer of carbon monoxide from the gas phase to liquid phase. Additionally, modulation can be obtained by changing the gas feed composition and thus the mole fraction of carbon monoxide in the gas feed.

Preferably the gas feed is introduced by the injector into the menstruum in the form of microbubbles having diameters in the range of 0.01 to 0.5, preferably 0.02 to 0.3 millimeter. At start-up and where desired, larger bubble sizes, in the range of 100 to 5000 microns in diameter may be used. Also a portion of the gas feed may be introduced by sparging to generate large bubbles, say, 1 to 5 or 10, millimeters in diameter, for assisting in mixing the aqueous menstruum. The gas substrate may be introduced into the bottom portion of the deep, bubble column reactor as a gas stream or as a gas in liquid dispersion as disclosed in U.S. patent application Ser. No. 12/826,991, filed Jun. 30, 2010. The presence of the oxygenated organic compound and/or other surface active agent enhances the formation of fine microbubbles.

The motive liquid may be any suitable liquid for introduction into the reactor. Advantageously, the motive liquid is one or more of aqueous menstruum, liquid derived from aqueous menstruum or make-up liquid to replace aqueous menstruum withdrawn from product recovery. Preferably the motive liquid comprises aqueous menstruum.

The flow rate of motive liquid used in an injector will depend upon the type, size and configuration of the injector and the sought bubble size of the gas feed. In general, the velocity of the dispersion stream leaving the injector is frequently in the range of 0.5 to 5 meters per second and the ratio of gas to motive liquid is in the range of from about 1:1 to 3:1 actual cubic meters per cubic meter of motive liquid.

The microbubbles form a stable gas-in-water dispersion. The introduction of the microbubbles into the aqueous menstruum places the microbubbles in a dynamic environment. The height of the aqueous menstruum means that microbubbles in the dispersion will experience different static pressure heads as they travel upwardly through the reactor. Increased pressure will, all else substantially the same, reduce the size of a microbubble. For a given gas feed rate, a greater surface area will be provided by the smaller microbubbles which enhances mass transfer. The size of a microbubble will also be affected by the diffusion of gases from the microbubble to the liquid phase. As carbon monoxide and hydrogen constitute a significant mole fraction of the microbubble as it is introduced into the aqueous menstruum, the dynamic conditions will promote a population of microbubbles that have small diameters to aid in maintaining the gas-in-water dispersion throughout the reactor.

The injectors may be located at one or more locations in the reactor and oriented in any suitable direction. Often the injectors are oriented to promote admixing of the gas feed with the aqueous menstruum and distribution in the reactor. The injectors may be located in a lower portion of the deep, MLD tank reactor. However, an advantage provided by using a deep, MLD tank reactor is that injectors may be placed at two or more heights. Due to the mechanical mixing, the dispersion introduced will be relatively uniform throughout the reactor and the average gas residence time will be advantageous to assure the sought transfer of carbon monoxide and hydrogen to the liquid phase. By locating the injectors over the height of the reactor, the uniformity of composition of the gas-in-liquid dispersion in the aqueous menstruum is promoted and less mechanical stifling energy may be required to maintain the sought uniformity.

### Gas Feed Composition and System Control

In accordance with the processes of this invention, a portion of the off-gas from above the top of the aqueous menstruum is admixed with fresh gas feed, or syngas, to enable high conversion of gas substrate to oxygenated organic compounds and to attenuate the risk of carbon monoxide inhibition of the microorganisms. The composition of the mixture will have a lower mole fraction of carbon monoxide and hydrogen than that in the syngas due to the presence of carbon dioxide contained in the recycle gas as well as inert or other gases that may be contained in the syngas such as nitrogen and methane. Since a portion of the off-gas is recycled, inert and other gases will build up to a steady-state composition.

The off-gases will contain some unreacted carbon monoxide and hydrogen. The portion of the off-gases that will be recycled to the aqueous menstruum will be sufficient to provide a molar conversion efficiency of total of carbon monoxide and hydrogen supplied with the syngas of at least about at least about 80, preferably at least about 85, often between about 85 and 95, percent. Accordingly, capital and operating costs associated with an additional reactor in series need not be incurred to provide commercially-attractive conversion efficiencies.

The operator can vary one or both of the recycle rate of off-gases and the feed rate of fresh syngas to achieve a desired conversion efficiency. For practical purposes, the injectors, especially slot injectors, have sufficient turn down capabilities that a wide range of gas volumes can be handled while still obtaining suitable microbubbles. Thus complex shutdown and start-up of injectors can be minimized, if not avoided, under steady-state operations.

The common commercial expectation is that the fermentation process will be operated to obtain a production rate of oxygenated organic compound that provides the greatest margin, i.e., the lowest fixed and variable cost per unit of production. As the cost of syngas is expected to be the primary cost driver, the operator has flexibility to operate the process to maximize margin as market conditions then exist. Aggressive production regimes can be used as the risk of carbon monoxide inhibition is attenuated by the recycle of off-gases. The operator can thus match the bioconversion capacity of the culture of microorganisms in the aqueous menstruum with the rate of transfer of carbon monoxide and hydrogen to the aqueous menstruum subject to equipment and energy limitations.

Due to this flexibility, the volume ratios of fresh syngas to recycled off-gases can vary widely. And these ratios will change should an event occur that adversely affects to productivity of the culture of microorganisms in the aqueous menstruum. The ratios are generally in the range of about 0.5:10 to 10:1, preferably, 1:5 to 5:1, cubic meter of recycled off-gas per cubic meter of fresh syngas at standard temperature and pressure. Frequently the gas feed compositions to the injectors are as set forth in the following table:

| Component | Usual, mole percent | Preferred, mole percent |
|---|---|---|
| Carbon monoxide | 5 to 50 | 10 to 35 |
| Hydrogen | 5 to 50 | 10 to 35 |
| Carbon dioxide | 10 to 70 | 10 to 50 |
| Nitrogen | 0 to 20 | 0 to 10 |
| Methane | 0 to 10 | 0 to 5 |

The gas feed may contain other components.

The portion of the off-gas not recycled, can be sent to recovery of any contained oxygenated organic compound and the remaining energy content recovered, e.g., by combustion in, for instance, a device such as a thermal oxidizer. The ratio of recycled to exhausted off-gas can vary widely depending upon the sought conversion of syngas to oxygenated organic compound. Practical limits exist to the conversion efficiencies that can be achieved in commercial operations. For instance, the exhaust stream should be sufficient to maintain inerts and other components in the off-gas and in the gas feed at acceptable levels.

A convenient control system for operating the processes of this invention involves determining the flow rate of the off-gas and analyzing the off-gas composition for carbon monoxide and hydrogen for comparison with the rate of carbon monoxide and hydrogen being provided by the fresh syngas to determine conversion efficiencies. The analysis may be conducted by any suitable method including but not limited to gas chromatography, mass spectroscopy, and infrared absorption as is well known. The rate of fresh syngas can be adjusted to achieve the targeted total conversion efficiency. Once steady-state operating conditions are achieved, the rate of off-gas recycle is typically maintained constant within the constraints of equipment limitations and so long as a major upset of the viability of the culture of microorganisms does not occur.

The recycled off-gases may be treated to remove a portion of the carbon dioxide prior to admixture with fresh syngas. Any suitable carbon dioxide removal process may be used including amine extraction, alkaline salt extractions, water absorption, membrane separation, adsorptions/desorption, and physical absorption in organic solvents. A preferred process for removal of carbon dioxide from gases is by contacting the gas with an aqueous solution containing oxygenated organic compound. This process for removing carbon dioxide from gas to be fed to a fermentation zone, including between sequential fermentation stages, is disclosed in U.S. Patent application No. 2008/0305539, filed Jul. 23, 2007, herein incorporated by reference in its entirety. See also, U.S. patent application Ser. No. 12/826,991, filed Jun. 30, 2010 herein incorporated by reference in its entirety, which discloses contacting a gas stream with a mixture of water and a surface active agent under pressure to sorb carbon dioxide and phase separating the gas and liquid stream to provide a gas stream with reduced carbon dioxide concentration to be used a feed to a fermentation zone. US 2008/0305539 A1 discloses the use of membranes to remove carbon dioxide from a membrane supported fermentation system to prevent dilution of concentrations of carbon monoxide and hydrogen in a multistage system.

If desired, a portion of the carbon dioxide dissolved in the liquid phase of the aqueous menstruum can be removed. Any convenient unit operation for carbon dioxide removal can be used, but the preferred operation is separation by reducing the pressure to atmospheric or lower pressure to flash carbon dioxide gas from the liquid phase.

### DRAWINGS

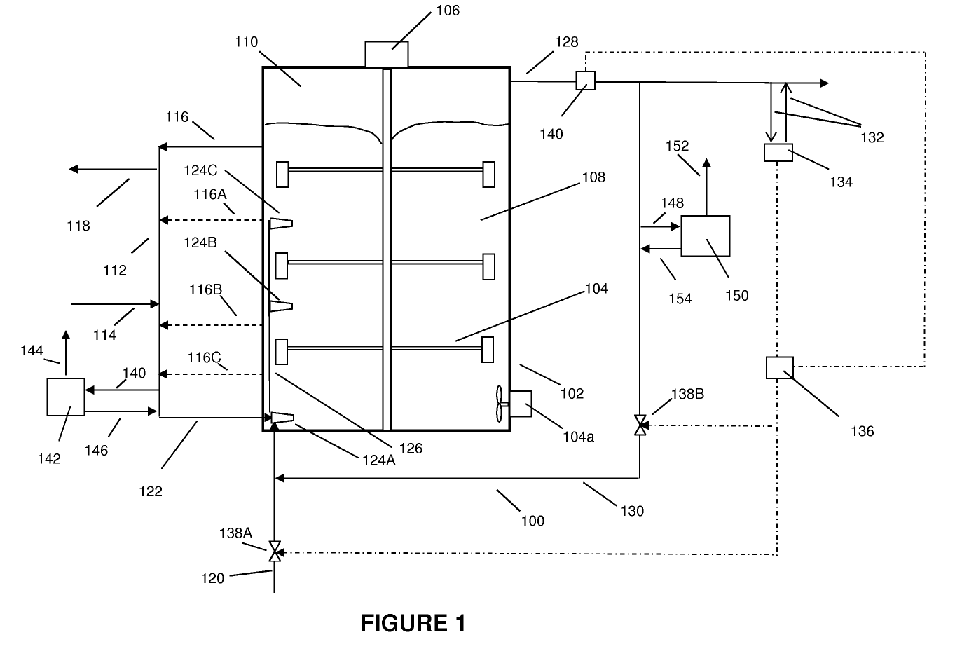

A general understanding of the invention and its application may be facilitated by reference to FIG. 1. FIG. 1 is a schematic depiction of an apparatus generally designated as 100 suitable for practicing the processes of this invention. FIG. 1 omits minor equipment such as pumps, compressors, valves, instruments and other devices the placement of which and operation thereof are well known to those practiced in chemical engineering. FIG. 1 also omits ancillary unit operations. The process and operation of FIG. 1 will be described in the context of the recovery and production of ethanol. The process is readily adaptable to making other oxygenated products such as acetic acid, butanol, propanol and acetone.

A continuous stirred tank fermentor assembly 100 comprising tank 102 having therein paddle agitator 104. Paddle agitator is shown as having three stifling paddle assemblies on a center shaft; however, fewer or more blades can be used. Motor 106 powers the agitator and controls the revolutions per minute. Alternatively, a plurality of side impellers could be used at different heights and orientations in the tank 102. For illustration, one side impeller 104a is depicted. Each side impeller may be separately oriented to provide the sought liquid mixing and the rotation of the impeller may be variable. An aqueous menstruum 108 is contained in tank 102. Above aqueous menstruum 108 in tank 102 is head space 110.

Aqueous menstruum is withdrawn from tank 102 via line 116 for product recovery and for recycle. As shown, line 116 is adapted to withdraw from the upper portion of liquid menstruum 108. The fermentor assembly 100 is adapted to operate at less than its full liquid capacity, e.g., during start-up operations. Lines 116A, 116B and 116C are provided to enable aqueous menstruum to be withdrawn from upper portions of lower volumes of aqueous menstruum. Each of lines 116, 116A, 116B and 116C are adapted to be in fluid flow communication with liquid header 112. Liquid header 112 is in fluid communication with line 118 for withdrawal of a portion of the aqueous menstruum for product recovery and purge. Line 114 provides make-up liquid to header 112. The make-up liquid provided by line 114 may be one or more of broth from a seed farm, recycle liquid from product recovery, and make-up water.

Syngas is provided to fermentor assembly 100 via line 120. The syngas is introduced in admixture with recycled off-gas as will be described below as a gas feed into aqueous menstruum 108 in the form of microbubbles. To achieve the microbubbles, gas feed and motive liquid, which is obtained from liquid header 112 and supplied by line 122, are passed to nozzles 124A, 124B and 124C. The motive liquid and gas feed are passed via line 126 to the other nozzles. Each nozzle may be a jet nozzle, or preferably, a slot injector. In a commercial scale unit more nozzles would be employed.

Off-gas from overhead zone 110 is removed via line 128. A portion of the removed off-gas may be treated to remove oxygenated organic compound and exhausted. Another portion of the removed gas is recycled to tank 102 via line 130.

The off-gas is analyzed to determine carbon monoxide and hydrogen compositions. As shown, gas analyzer 134 is located in communication with line 128 via lines 132 to withdraw and return gas samples. Analyzer 134, for purposes of this depiction is a gas chromatograph/mass spec. Analyzer 134 is in data communication with control processor 136 which is a computer containing algorithms to determine the conversion efficiency of carbon monoxide and hydrogen in the fresh syngas. Control processor 136 is also in data communication with flow meter 140 which is adapted to determine the off-gas flow rate from tank 102. Control processor 136 is in data communication with valve 138A in line 120 to adjust the rate of fresh syngas supply to obtain the targeted conversion efficiency. In the event that the analysis indicates that the rate of recycle needs to be adjusted, control processor 136 is also in data communication with valve 138B.

Assembly 100 is provided with a unit operation to remove carbon dioxide from the aqueous menstruum. As shown, recycling aqueous menstruum in header 112 is withdrawn via line 140 and passed to flash tank 142. Flash tank 142 is maintained under a lower pressure, usually about ambient atmospheric pressure, and thus carbon dioxide effervesces and is removed via line 144. The aqueous menstruum with a reduced carbon dioxide content is returned to header 112 via line 146.

Carbon dioxide can also be removed from the recycling off-gas. As shown, recycling off-gas is withdrawn from line 130 via line 148 and passes to carbon dioxide removal unit operation 150. Carbon dioxide removal unit operation 150 may be any suitable device. For instance, carbon dioxide can be removed by sorption into an aqueous stream containing ethanol and the sorbent then regenerated to yield carbon dioxide which is removed via line 152. The recycling off-gas with a reduced carbon dioxide concentration is returned to line 130 via line 154.

It is claimed:

1. A process for bioconverting CO, H2 and CO2 to oxygenated organic compound comprising:
a. passing a gas feed comprising CO, H2 and CO2 into in a reactor containing aqueous menstruum under aerobic fermentation conditions, said aqueous menstruum containing microorganisms adapted for bioconverting syngas to oxygenated organic compound, to produce oxygenated organic compound dissolved in the aqueous menstruum and an off gas;
b. maintaining in said primary reactor a depth of aqueous menstruum of at least 10 meters;
c. maintaining in said reactor a head space above the upper portion of the aqueous menstruum;
d. continuously supplying the gas feed to said aqueous menstruum through a plurality of injectors that use a motive liquid to form a stable gas-in-liquid dispersion in at least a lower portion of the aqueous menstruum;
e. modulating the microbubble size to control the rate of transfer of the carbon monoxide and hydrogen to aqueous menstruum and provide a stable gas-in-liquid dispersion; and,
f. bioconverting carbon monoxide and hydrogen and carbon dioxide to an oxygenated organic compound and providing off-gas from the aqueous menstruum in the head space.

2. The process of claim 1 wherein the oxygenated compound is at least one of ethanol, acetic acid, propanol, propionic acid, butanol and butyric acid.

3. The process of claim 1 wherein the reactor has an aspect ratio of height to diameter of between about 0.5:1 to 5:1.

4. The process of claim 1 wherein the volume of aqueous menstruum in the reactor is at least about 1 million liters.

5. The process of claim 1 wherein the microbubbles are between about 10 and 500 microns in diameter.

6. The process of claim 1 the reactor is in a start-up mode and the microbubbles have a diameter in the range of 100 to 5000 microns.

7. The process of claim 1 wherein the rate of flow of the motive liquid adjusts the size of the microbubbles to provide an interfacial surface area between the gas phase and liquid phase to provide a rate of transfer of carbon monoxide and hydrogen that is low enough to avoid carbon monoxide inhibition.

8. The process of claim 1 wherein the rate of supply of gas substrate for admixing with the recycled off-gas is controlled in response to the conversion efficiency.

9. The process of claim 1 wherein the motive liquid comprises aqueous menstruum.

10. The process of claim 1 wherein the gas feed is supplied at two or more heights in the reactor.

11. The process of claim 2 wherein between about 1:5 to 5:1 cubic meter of recycle gases are recycled per cubic meter of fresh gas substrate at standard temperature and pressure.

12. The process of claim 11 wherein the admixture of gas substrate and recycled off-gas comprises about 5 to 50 mole percent carbon monoxide, about 5 to 50 mole percent hydrogen, and about 10 to 70 mole percent carbon dioxide.

13. The process of claim 12 wherein the conversion of the total moles of carbon monoxide and hydrogen in the gas substrate to oxygenated organic compound of at least about 85 percent.

14. The process of claim 2 wherein the time for distribution in the reactor is less than 25 percent of the residence time of the gas in the reactor.

15. The process of claims 1 wherein the injectors are jet injectors and have a cross-sectional dimension of at least about 1 to 4 centimeters.

16. The process of claim 1 wherein the injectors are jet injectors are slot injectors and the smaller cross-sectional dimension of the slot is at least about 1 to 4 centimeters.

17. The process of claim 1 wherein the modulation is obtained by at least one of changing (i) the gas to liquid flow ratio to the injector thus changing the volume of gas feed and (ii) the rate of motive liquid and the resulting bubble size, and (iii) the mole fraction of carbon monoxide in the gas feed.

18. The process of claim 1 wherein the velocity of the dispersion stream discharged from the ejector is in the range of 0.5 to 5 meters per second and the ratio of gas to motive liquid is in the range of from about 1:1 to 3:1 actual cubic meters per cubic meter of motive liquid.

19. The process of claim 1 wherein the average residence time of the gas feed in the reactor is between about 100 and 300 seconds.

20. The process of claim 1 wherein: a control processor communicates with a gas analyzer that is in fluid communication with the head space and is adapted to determine the concentration of carbon monoxide and hydrogen in the off-gas from the aqueous menstruum; the control processor communicates with a flow meter adapted to determine the flow rate of off-gas being produced; the control processor adapted determines the conversion efficiency of carbon monoxide and hydrogen in the tank reactor; and processor controls the rate of flow of fresh gas feed to the reactor in response to the conversion efficiency of carbon monoxide and hydrogen.
